(19) United States
(12) Patent Application Publication
(10) Pub. No.: US 2012/0021919 A1
(43) Pub. Date: Jan. 26, 2012

(54) Identification of Differentially Represented Fetal or Maternal Genomic Regions and Uses Thereof

(76) Inventors: Thomas Scholl, Westborough, MA (US); Viatcheslav R. Akmaev, Brookline, MA (US)

(21) Appl. No.: 13/188,794

(22) Filed: Jul. 22, 2011

Related U.S. Application Data
(60) Provisional application No. 61/367,254, filed on Jul. 23, 2010.

(51) Int. Cl. C40B 20/00; C40B 30/04; G01N 33/53; C12Q 1/68

(52) U.S. Cl. 506/2; 435/6.12; 435/6.11; 506/9; 436/501

(57) ABSTRACT

The present invention provides a novel approach for identification and characterization of differentially represented fetal or maternal genomic regions in maternal circulation. Identification of overrepresented fetal genomic regions in the maternal circulation according to the present invention permit accurate analysis of fetal DNA without the need for enrichment or purification, which provides a simpler, more accurate and efficient prenatal diagnosis in early pregnancy. The present invention is particularly useful for noninvasive prenatal diagnosis during early pregnancy (e.g., during the first trimester).

### RELATED APPLICATIONS

This application claims priority to U.S. Provisional Application No. 61/367,254 filed Jul. 23, 2010. The disclosure of U.S. Provisional Application No. 61/367,254 is incorporated by reference in its entirety herein.

### BACKGROUND

Molecular analysis of cell free fetal DNA in maternal circulation has been shown to be a promising approach in non-invasive prenatal diagnosis of fetal aneuploidy, other fetal genetic abnormalities and pregnancy complications. Many existing diagnostic methods and techniques typically perform well in clinical cases where the fraction of cell free fetal DNA in maternal plasma exceeds 25%. However, such levels of fetal DNA are typically reached only late in pregnancy when a therapeutic intervention is no longer an option. It has been observed that the fraction of cell free fetal DNA in maternal plasma varies between 0% to 5-10% in the first trimester of pregnancy between 9 and 13 weeks of gestation. To reach clinically useful accuracy in the first trimester of pregnancy, a significant enrichment of the fetal material is usually required for any of the currently developed assays.

### SUMMARY OF THE INVENTION

The present invention provides a novel approach for identification and characterization of differentially represented (e.g., overrepresented or underrepresented) fetal or maternal genomic regions in maternal circulation. Among other things, identification of overrepresented fetal genomic regions in maternal circulation according to the present invention may permit accurate analysis of fetal DNA without enrichment or purification, resulting in simpler, more accurate and efficient pre-natal diagnostic assays. The present invention is particularly useful for noninvasive pre-natal diagnosis during early pregnancy (e.g., during the first trimester).

In some embodiments, the present invention provides a method of identifying differentially represented fetal or maternal genomic regions in a maternal sample, comprising steps of quantifying a fetal or maternal genomic region present in a maternal sample; determining relative abundance of the fetal or maternal genomic region as compared to a reference amount, thereby determining if the fetal or maternal genomic region is differentially represented in the maternal sample; wherein the fetal or maternal genomic region does not correspond to an aneuploidic region.

In some embodiments, a reference amount is indicative of an average representation of fetal or maternal nucleic acid in a maternal sample. In some embodiments, the step of determining relative abundance comprises comparing the quantified amount to the reference amount and further wherein the fetal or maternal genomic region is identified as differentially represented in the maternal sample if the quantified amount is different than the reference amount with statistical confidence.

In some embodiments, a reference amount is indicative of an overrepresentation of fetal or maternal nucleic acid in a maternal sample. In some embodiments, the step of determining relative abundance comprises comparing the quantified amount to the reference amount and further wherein the fetal or maternal genomic region is identified as overrepresented in the maternal sample if the quantified amount is substantially the same as or greater than the reference amount with statistical confidence.

In some embodiments, a reference amount is indicative of an underrepresentation of fetal or maternal nucleic acid in a maternal sample. In some embodiments, the step of determining relative abundance comprises comparing the quantified amount to the reference amount and further wherein the fetal or maternal genomic region is identified as underrepresented in the maternal sample if the quantified amount is substantially the same as or less than the reference amount with statistical confidence.

In some embodiments, a method according to the present invention quantifies a fetal genomic region. In some embodiments, the reference amount is indicative of an average representation of fetal nucleic acid in the maternal sample. In some embodiments, an average representation of fetal nucleic acid is 5%. In some embodiments, a fetal genomic region is identified as overrepresented in the maternal sample if the amount quantified is above the reference amount with statistical confidence.

In some embodiments, a method according to the present invention quantifies a maternal genomic region. In some embodiments, the reference amount is indicative of an average representation of maternal nucleic acid in the maternal sample. In some embodiments, an average representation of maternal nucleic acid is 95%. In some embodiments, a maternal genomic region is identified as underrepresented in the maternal sample if the amount quantified is below the reference amount with statistical confidence.

In some embodiments, the quantifying step of a method according to the invention comprises quantifying a fetal genomic region and the corresponding maternal genomic region. In some embodiments, the relative abundance of the fetal genomic region is determined by comparing the quantified amount of the fetal genomic region to the quantified amount of the corresponding maternal genomic region. In some embodiments, a fetal genomic region is distinctively detectable from the corresponding maternal genomic region. In some embodiments, a fetal genomic region contains a paternally contributed sequence. In some embodiments, a fetal genomic region contains a sequence distinct from the corresponding maternal genomic region. In some embodiments, a fetal genomic region contains at least one polymorphic nucleotide distinct from the corresponding maternal genomic region. In some embodiments, a fetal genomic region contains a methylation pattern that is distinct from the corresponding maternal genomic region. In some embodiments, a fetal genomic region contains copy number variation (CNV) as compared to the corresponding maternal genomic region.

In some embodiments, a method according to the invention is performed in a high throughput format. In some embodiments, a method according to the invention quantifies multiple fetal or maternal genomic regions simultaneously.

In some embodiments, a method according to the invention further includes a step of first preparing total DNA from the maternal sample. In some embodiments, a method according to the invention further includes a step of first preparing cell free DNA from the maternal sample. In some embodiments, a method according to the invention further includes a step of first generating nucleic acid fragments containing the fetal or maternal genomic region to be quantified.

In some embodiments, a maternal sample suitable for the present invention is selected from the group consisting of cells, tissue, whole blood, plasma, serum, urine, stool, saliva, cord blood, chorionic villus sample, chorionic villus sample culture, amniotic fluid, amniotic fluid culture, transcervical lavage fluid, and combination thereof. In particular embodiments, a maternal sample suitable for the invention is maternal blood.

In some embodiments, a maternal sample suitable for the invention is obtained from one individual. In some embodiments, a maternal sample suitable for the invention is obtained from multiple individuals.

In some embodiments, the quantifying step of a method according to the invention includes a DNA sequencing step. In some embodiments, the DNA sequencing step includes a high-throughput single molecule sequencing step. In some embodiments, the DNA sequencing step includes an unbiased DNA sequencing step. In some embodiments, the DNA sequencing step cover greater than 1, 5, 10, 20, 30, 40, 50, 60, 70, 80, 90, or 100 genomic equivalence.

In some embodiments, the DNA sequencing step includes a step of labeling the fetal or maternal genomic region with optical signal. In some embodiments, the optical signal is selected from fluorescent and/or luminescent signal. In some embodiments, the fluorescent signal is generated by Cyanine-3 and/or Cyanine-5.

In some embodiments, a method of the invention further includes a step of capturing nucleic acid molecules (e.g., nucleic acid fragments) containing the fetal or maternal genomic region to be quantified onto a solid surface prior to the sequencing step.

In some embodiments, a quantifying step according to the invention involves obtaining individual sequence read counts attributable to the fetal or maternal genomic region. In some embodiments, a quantifying step according to the invention further involves comparing the individual sequence read counts attributable to the fetal genomic region to the individual sequence read counts attributable to the corresponding maternal genomic region.

In some embodiments, a quantifying step according to the invention includes a step of performing digital PCR.

In some embodiments, a quantifying step according to the invention includes a step of performing bridge PCR.

In some embodiments, a quantifying step according to the invention includes a step of hybridizing individual nucleic acid molecules using probes labeled with nanoreporters that specifically bind to the fetal or maternal genomic region. Nanoreporters according to embodiments of the present invention are described in U.S. Patent Publication No. 20100047924, the contents of which are incorporated herein.

In some embodiments, a quantifying step according to the invention includes a step of performing array-based comparative genomic hybridization (aCGH). In some embodiments, the aCGH step uses probes that specifically bind to the fetal or maternal genomic region. In some embodiments, the probes are labeled with optical signal. In some embodiments, the optical signal is selected from fluorescent and/or luminescent signal. In some embodiments, the aCGH step involves determining the level of signal attributable to the fetal or maternal genomic region.

In some embodiments, the statistical confidence used in a method according to the invention is determined by N-way ANOVA, Student t-test, Fisher's exact test, or multiple testing corrections.

In some embodiments, a method of the invention further includes a step of determining an overrepresentation factor of the fetal genomic region.

In some embodiments, a method of the invention further comprises comparing the identified differentially represented fetal or maternal genomic region across different individuals. In some embodiments, a method of the invention further include a step of validating the identified differentially represented fetal or maternal genomic region (e.g., by digital PCR or resequencing).

In certain embodiments, the present invention provides a method of identifying fetal genomic regions normally overrepresented in a maternal sample, comprising steps of characterizing a fetal genomic region and corresponding maternal genomic region in a maternal sample; determining relative abundance of the fetal genomic region as compared to the corresponding maternal genomic region; and identifying the fetal genomic region as overrepresented in the maternal sample if the relative abundance determined is above a predetermined threshold with statistical confidence, wherein the fetal genomic region is not an aneuploidic region.

In certain embodiments, the present invention provides a method of identifying maternal genomic regions normally underrepresented in a maternal sample, comprising steps of characterizing a maternal genomic region and corresponding fetal genomic region in a maternal sample; determining relative abundance of the maternal genomic region as compared to the corresponding fetal genomic region; and identifying the maternal genomic region as underrepresented in the maternal sample if the relative abundance determined is below a predetermined threshold with statistical confidence, wherein the corresponding fetal genomic region is not an aneuploidic region.

In certain embodiments, the present invention provides a method of identifying fetal genomic regions normally overrepresented in a maternal sample, comprising steps of characterizing a fetal genomic region in a maternal sample; determining relative abundance of the fetal genomic region as compared to a reference; and identifying the fetal genomic region as overrepresented in the maternal sample if the relative abundance determined is above a pre-determined threshold with statistical confidence, wherein the fetal genomic region is not an aneuploidic region. In particular embodiments, the reference suitable for the present invention is indicative of an average representation of fetal nucleic acid in a maternal sample.

In certain embodiments, the present invention provides a method of identifying maternal genomic regions normally underrepresented in a maternal sample, comprising steps of characterizing a maternal genomic region in a maternal sample; determining relative abundance of the maternal genomic region as compared to a reference; and identifying the maternal genomic region as underrepresented in the maternal sample if the relative abundance determined is below a pre-determined threshold with statistical confidence, wherein the maternal genomic region does not correspond to an aneuploidic region. In particular embodiments, the reference suitable for the present invention is indicative of an average representation of maternal nucleic acid in a maternal sample.

In some embodiments, the present invention also provides various methods of noninvasive diagnosis including a step of characterizing an overrepresented fetal genomic region identified using a method described herein.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### DEFINITIONS

In order for the present invention to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Allele: As used herein, the phrase “allele” is used interchangeably with “allelic variant” and refers to a variant of a locus or gene. In some embodiments, different alleles or allelic variants are polymorphic.

Amplification: As used herein, the term “amplification” refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100 fold difference). Amplification by asymmetric PCR is generally linear. A skilled artisan will understand that different amplification methods may be used together.

Aneuploidy: As used herein, the term “aneuploidy” refers to an abnormal number of whole chromosomes or parts of chromosomes. Typically, aneuploidy causes a genetic imbalance which may be lethal at early stages of development, cause miscarriage in later pregnancy or result in a viable but abnormal pregnancy. The most frequent and clinically significant aneuploidies involve single chromosomes (strictly “aneusomy”) in which there are either three (“trisomy”) or only one (“monosomy”) instead of the normal pair of chromosomes.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biological sample: As used herein, the term “biological sample” encompasses any sample obtained from a biological source. In certain embodiments, a biological source is a subject. A biological sample can, by way of non-limiting example, include blood, amniotic fluid, sera, urine, feces, epidermal sample, skin sample, cheek swab, sperm, amniotic fluid, cultured cells, bone marrow sample and/or chorionic villi from a subject. Convenient biological samples may be obtained by, for example, scraping cells from the surface of the buccal cavity. Cell cultures of any biological samples can also be used as biological samples, e.g., cultures of chorionic villus samples and/or aminoitic fluid cultures such as amniocyte cultures. A biological sample can also be, e.g., a sample obtained from any organ or tissue (including a biopsy or autopsy specimen), can comprise cells (whether primary cells or cultured cells), medium conditioned by any cell, tissue or organ, tissue culture. In some embodiments, biological samples suitable for the invention are samples which have been processed to release or otherwise make available a nucleic acid for detection as described herein. Suitable biological samples may be obtained from a stage of life such as a fetus, young adult, adult (e.g., pregnant women), and the like. Fixed or frozen tissues also may be used. The terms “biological sample” and “biological specimen” are used interchangeably.

Copy number: As used herein, the phrase “copy number” when used in reference to a locus, refers to the number of copies of such a locus present per genome or genome equivalent. A “normal copy number” when used in reference to a locus, refers to the copy number of a normal or wild-type allele present in a normal individual. In certain embodiments, the copy number ranges from zero to two inclusive. In certain embodiments, the copy number ranges from zero to three, zero to four, zero to six, zero to seven, or zero to more than seven copies, inclusive. In embodiments in which the copy number of a locus varies greatly across individuals in a population, an estimated median copy number could be taken as the “normal copy number” for calculation and/or comparison purposes.

Corresponding fetal or maternal genomic regions: As used herein, the term “corresponding fetal or maternal genomic regions” refers to genomic regions from fetal or maternal nucleic acids but mapped to the same chromosomal location.

Complement: As used herein, the terms “complement,” “complementary” and “complementarity,” refer to the pairing of nucleotide sequences according to Watson/Crick pairing rules. For example, a sequence 5′-GCGGTCCCA-3′ has the complementary sequence of 5′-TGGGACCGC-3′. A complement sequence can also be a sequence of RNA complementary to the DNA sequence. Certain bases not commonly found in natural nucleic acids may be included in the complementary nucleic acids including, but not limited to, inosine, 7-deazaguanine, Locked Nucleic Acids (LNA), and Peptide Nucleic Acids (PNA). Complementary need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control. In some embodiments, a control is also referred to as a reference.

Crude: As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

Differentially represented: As used herein, the term “differentially represented” refers to a level of representation of a genomic region (e.g., fetal or maternal) that is deviate from the baseline. Typically, the baseline is indicative of an average representation of fetal or genomic nucleic acid in maternal circulation (e.g., maternal blood). A differentially represented region can be an over represented or under represented region. As used herein, the term “overrepresented” or “over representation” refers to a level of representation of a genomic region that is substantially above the baseline with statistic confidence. As used herein, the term “under represented” or “under representation” refers to a level of representation of a genomic region that is substantially below the baseline with statistic confidence.

Deletion: As used herein, the term “deletion” encompasses a mutation that removes one or more nucleotides from a naturally-occurring nucleic acid.

Gene: As used herein, the term “gene” refers to a discrete nucleic acid sequence responsible for a discrete cellular (e.g., intracellular or extracellular) product and/or function. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. As used herein, the term “gene” can also include nucleic acids that do not encode proteins but rather provide templates for transcription of functional RNA molecules such as tRNAs, rRNAs, etc. Alternatively, a gene may define a genomic location for a particular event/function, such as a protein and/or nucleic acid binding site.

Genotype: As used herein, the term “genotype” refers to the genetic constitution of an organism. More specifically, the term refers to the identity of alleles present in an individual. Genotyping is the process of elucidating the genotype of an individual with a biological assay. Genotyping of an individual or a DNA sample typically refers to identifying the nature, in terms of nucleotide base, of the two alleles possessed by an individual at a known polymorphic site.

Hybridize: As used herein, the term “hybridize” or “hybridization” refers to a process where two complementary nucleic acid strands anneal to each other under appropriately stringent conditions. Oligonucleotides or probes suitable for hybridizations typically contain 10-100 nucleotides in length (e.g., 18-50, 12-70, 10-30, 10-24, 18-36 nucleotides in length). Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

Individually resolved: As used herein, the term “individually resolved” is used herein to indicate that, when visualised, it is possible to distinguish one polymer or clone from its neighbouring polymers or clones. Visualisation may be effected by the use of reporter labels, e.g. fluorophores, the signal of which is individually resolved. The requirement for individual resolution ensures that individual monomer incorporation can be detected at each synthesis step.

Insertion or addition: As used herein, the term “insertion” or “addition” refers to a change in an amino acid or nucleotide sequence resulting in the addition of one or more amino acid residues or nucleotides, respectively, as compared to the naturally occurring molecule.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Labeled: The terms “labeled” and “labeled with a detectable agent or moiety” are used herein interchangeably to specify that an entity (e.g., a nucleic acid probe, antibody, etc.) can be visualized, for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, colorimetric labels, magnetic labels, haptens, molecular beacons, aptamer beacons, and the like.

Locus: As used herein, the term “locus” refers to the specific location of a particular DNA sequence on a chromosome. As used herein, a particular DNA sequence can be of any length (e.g., one, two, three, ten, fifty, or more nucleotides). In some embodiments, the locus is or comprises a gene or a portion of a gene. In some embodiments, the locus is or comprises an exon or a portion of an exon of a gene. In some embodiments, the locus is or comprises an intron or a portion of an intron of a gene. In some embodiments, the locus is or comprises a regulatory element or a portion of a regulatory element of a gene. In some embodiments, the locus is associated with a disease, disorder, and/or condition. For example, mutations at the locus (including deletions, insertions, splicing mutations, point mutations, etc.) may be correlated with a disease, disorder, and/or condition.

Karyotyping: As used herein, the term “karyotyping” encompasses a determination of the number of chromosomes in a eukaryote cell.

Maternal sample: As used herein, the term “maternal sample” refers to a biological sample obtained from a pregnant woman. See the definition of Biological Sample.

Normal: As used herein, the term “normal,” when used to modify the term “copy number” or “locus” or “gene” or “allele,” refers to the copy number or locus, gene, or allele that is present in the highest percentage in a population, e.g., the wild-type number or allele. When used to modify the term “individual” or “subject” they refer to an individual or group of individuals who carry the copy number or the locus, gene or allele that is present in the highest percentage in a population, e.g., a wild-type individual or subject. Typically, a normal “individual” or “subject” does not have a particular disease or condition and is also not a carrier of the disease or condition. The term “normal” is also used herein to qualify a biological specimen or sample isolated from a normal or wild-type individual or subject, for example, a “normal biological sample.”

Multiplex PCR: As used herein, the term “multiplex PCR” refers to amplification of two or more regions which are each primed using a distinct primers pair.

Primer: As used herein, the term “primer” refers to a short single-stranded oligonucleotide capable of hybridizing to a complementary sequence in a nucleic acid sample. Typically, a primer serves as an initiation point for template dependent DNA synthesis. Deoxyribonucleotides can be added to a primer by a DNA polymerase. In some embodiments, such deoxyribonucleotides addition to a primer is also known as primer extension. The term primer, as used herein, includes all forms of primers that may be synthesized including peptide nucleic acid primers, locked nucleic acid primers, phosphorothioate modified primers, labeled primers, and the like. A “primer pair” or “primer set” for a PCR reaction typically refers to a set of primers typically including a “forward primer” and a “reverse primer.” As used herein, a “forward primer” refers to a primer that anneals to the anti-sense strand of dsDNA. A “reverse primer” anneals to the sense-strand of dsDNA.

Polymorphism: As used herein, the term “polymorphism” refers to the coexistence of more than one form of a gene or portion thereof.

Probe: As used herein, the term “probe,” when used in reference to a probe for a nucleic acid, refers to a nucleic acid molecule having specific nucleotide sequences (e.g., RNA or DNA) that can bind or hybridize to nucleic acids of interest. Typically, probes specifically bind (or specifically hybridize) to nucleic acid of complementary or substantially complementary sequence through one or more types of chemical bonds, usually through hydrogen bond formation. In some embodiments, probes can bind to nucleic acids of DNA amplicons in a real-time PCR reaction.

Relative abundance: As used herein, the term “relative abundance” refers to an amount of a genomic region of interest as compared to a reference amount. Any appropriate reference amount can be used to determine the relative abundance of a genomic region of interest. See, the definition of Reference Amount. Typically, relative abundance encompasses ratios between the amount of two genomic regions (e.g., fetal DNA vs. the corresponding maternal genomic DNA), percentages (e.g., the percentage of fetal DNA out of the total amount of DNA), change of fold, normalized amount, among others. The term “relative abundance” is used inter-changeably with “relative amount.”

Reference amount: As used herein, the term “reference amount” refers to any amount that can be used as a comparison standard or control to calculate the relative abundance of a genomic region of interest. In general, a reference amount can be an amount indicative of a total amount, an average amount, an overrepresented, or underrepresented amount. For example, a reference amount can be an amount indicative of the total amount of nucleic acid in a relevant maternal sample (e.g., maternal blood), the total amount of fetal nucleic acid, the total amount of maternal nucleic acid, the amount of a control region which is known not to be over or under represented or an average amount of multiple control regions, the amount of a known overrepresented region or an average amount of multiple overrepresented regions, the amount of a known underrepresented region or an average amount of multiple overrepresented regions, or the amount of the genomic region (e.g., fetal or maternal) corresponding to the region of interest. A reference amount can be an amount obtained from a quantifying reaction or assay that is performed simultaneously with the region of interest to provide a comparator; a historical reference (i.e., an amount or result from an assay performed previously, or an amount or result that is previously known); a printed or otherwise saved record; or a pre-determined threshold. In some embodiments, a reference amount is indicative of the average representation of fetal nucleic acid in maternal blood (e.g., 3%, 5%, 10%, 15%, or 20%). In some embodiments, a reference amount is indicative of the average representation of maternal nucleic acid in maternal blood (e.g., 97%, 95%, 90%, 85%, or 80%).

Sense strand vs. anti-sense strand: As used herein, the term “sense strand” refers to the strand of double-stranded DNA (dsDNA) that includes at least a portion of a coding sequence of a functional protein. As used herein, the term “anti-sense strand” refers to the strand of dsDNA that is the reverse complement of the sense strand.

Signal: As used herein, the term “signal” refers to a detectable and/or measurable entity. In certain embodiments, the signal is detectable by the human eye, e.g., visible. For example, the signal could be or could relate to intensity and/or wavelength of color in the visible spectrum. Non-limiting examples of such signals include colored precipitates and colored soluble products resulting from a chemical reaction such as an enzymatic reaction. In certain embodiments, the signal is detectable using an apparatus. In some embodiments, the signal is generated from a fluorophore that emits fluorescent light when excited, where the light is detectable with a fluorescence detector. In some embodiments, the signal is or relates to light (e.g., visible light and/or ultraviolet light) that is detectable by a spectrophotometer. For example, light generated by a chemiluminescent reaction could be used as a signal. In some embodiments, the signal is or relates to radiation, e.g., radiation emitted by radioisotopes, infrared radiation, etc. In certain embodiments, the signal is a direct or indirect indicator of a property of a physical entity. For example, a signal could be used as an indicator of amount and/or concentration of a nucleic acid in a biological sample and/or in a reaction vessel.

Specific: As used herein, the term “specific,” when used in connection with an oligonucleotide primer, refers to an oligonucleotide or primer, under appropriate hybridization or washing conditions, is capable of hybridizing to the target of interest and not substantially hybridizing to nucleic acids which are not of interest. Higher levels of sequence identity are preferred and include at least 60%, 65%, 70%, 75%, 80%, 85%, 90%, 95%, 98%, 99%, or 100% sequence identity. In some embodiments, a specific oligonucleotide or primer contains at least 4, 6, 8, 10, 12, 14, 16, 18, 20, 22, 24, 26, 28, 30, 35, 40, 45, 50, 55, 60, 65, 70, or more bases of sequence identity with a portion of the nucleic acid to be hybridized or amplified when the oligonucleotide and the nucleic acid are aligned.

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially complementary: As used herein, the term “substantially complementary” refers to two sequences that can hybridize under stringent hybridization conditions. The skilled artisan will understand that substantially complementary sequences need not hybridize along their entire length. In some embodiments, “stringent hybridization conditions” refer to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 533 Denhart's solution at 42° C. overnight; washing with 2×SSC, 0.1% SDS at 45° C.; and washing with 0.2×SSC, 0.1% SDS at 45° C. In some embodiments, stringent hybridization conditions should not allow for hybridization of two nucleic acids which differ over a stretch of 20 contiguous nucleotides by more than two bases.

Substitution: As used herein, the term “substitution” refers to the replacement of one or more amino acids or nucleotides by different amino acids or nucleotides, respectively, as compared to the naturally occurring molecule.

Wild-type: As used herein, the term “wild-type” refers to the typical or the most common form existed in nature.

### DETAILED DESCRIPTION

The present invention provides, among other things, methods of identifying and characterizing differentially represented (e.g., overrepresented or underrepresented) fetal or maternal genomic regions in maternal circulation. The invention encompasses the recognition that certain genomic regions from the fetus DNA may normally be over represented in maternal circulation and identification of such over represented fetal genomic regions may allow accurate pre-natal diagnosis based on such over represented regions without significant enrichment or purification of fetal DNA. It is contemplated that over representation of fetal genomic regions may be caused by multiple factors such as DNA structure, specifics of the cell, DNA break-up process during apoptosis, and DNase accessibility in blood.

Typically, a method according to the present invention involves quantifying one or more fetal or maternal genomic regions of interest present in maternal circulation and determining relative abundance of individual fetal or maternal genomic regions as compared to an appropriate reference amount. Various reference amounts can be used to determine relative abundance. In some embodiments, a reference amount indicative of average representation of fetal or maternal nucleic acid in maternal circulation is used to determine relative abundance and a genomic region is identified as differentially represented if the relative abundance of the genomic region is different than the reference amount with statistical confidence. A reference amount indicative of over or under representation may also be used to determine relative abundance.

Typically, a differentially (e.g., under or over) represented region identified according to the present invention does not correspond to an aneuploidic region.

Differentially represented regions, in particular, relatively overrepresented fetal genomic regions, can be used to develop pre-natal diagnostic assays without requiring significant fetal DNA enrichment or purification. In certain embodiments, a relatively overrepresented fetal genomic region useful for pre-natal diagnosis is identified based on at least the following two qualities: (1) the overrepresentation of the normalized amount of the fetal genomic region in maternal circulation as compared to other fetal regions; and/or (2) the split (i.e., the ratio) between the fetal genomic region and the corresponding maternal region. With respect to the latter quality, it is contemplated that relative over representation of certain fetal genomic regions in maternal circulation may be a result of a relative under representation of the corresponding maternal regions. An analysis of these two qualities may demonstrate, for example, that a particular fetal genomic region is relatively overrepresented compared to corresponding maternal region, but may be relatively underrepresented as compared to other fetal genomic regions. Ideally, a fetal genomic region used in a prenatal diagnostic assay is relatively overrepresented compared to the corresponding maternal region and relatively overrepresented as compared to other fetal genomic regions.

Various aspects of the invention are described in detail in the following sections. The use of sections is not meant to limit the invention. Each section can apply to any aspect of the invention. In this application, the use of “or” means “and/or” unless stated otherwise.

### Identification of Polymorphic Regions

To facilitate accurate determination of differentially represented fetal or maternal genomic regions, a method of the invention typically utilizes a characterization assay that can distinguish between a fetal genomic region and the corresponding maternal genomic region. Accordingly, in some embodiments, the present invention involves a step of first identifying those fetal genomic regions that are distinctively detectable from their corresponding maternal genomic regions. This step is also referred to as a step of identifying polymorphic regions. As used herein, the term “polymorphic regions” encompasses both those regions containing sequence variations (such as SNPs) and regions with identical sequences but otherwise distinctively detectable due to epigenetic modification (such as methylation).

Typically, fetal genomic regions that are distinctively detectable from their corresponding maternal regions contain paternally contributed sequences. In some embodiments, paternally contributed sequences (or information derived therefrom) serve as markers of fetal nucleic acids (or information derived therefrom). For example, descriptions of methods comprising comparing fetal nucleic acids with maternal nucleic acids are intended to encompass embodiments in which paternally contributed nucleic acids are compared to maternal nucleic acids. In embodiments where paternally contributed nucleic acids are analyzed or used, paternally contributed nucleic acids are intended to encompass fetal nucleic acids. In some embodiments, a fetal genomic region is distinctively detectable because it contains a sequence that is distinct from the corresponding maternal genomic region (e.g., one or more polymorphic nucleotides). In some embodiments, a fetal genomic region is distinctively detectable because it contains copy number variations (CNVs) as compared to the corresponding maternal region. In some embodiments, a fetal genomic region is distinctively detectable because it contains a methylation pattern or other epigenetic modification that is distinct from the corresponding maternal genomic region. Methods of detecting methylation are known in the art and can be adapted for use in accordance with the present invention. Typically, to detect distinct methylation patterns, nucleic acids may be treated to convert methylated and unmethylated nucleotides into distinct nucleotides. For example, in some DNA methylation detection assays, nucleic acids are treated with an agent that converts unmethylated guanine bases but not methylated guanine bases, or vice versa. For example, sodium bisulfite converts unmethylated guanines to thymines but does not convert methylated guanines. Thus, methylation can be detected by treating nucleic acids (e.g., DNA) with such agents and then performing one or more techniques to determine the sequence of the treated nucleic acid, thereby determining whether one or more guanosines in the nucleic acid was methylated. For example, sodium bisulfate treatment may be combined with a sequencing method (e.g., single molecule sequencing), or primer extension method in order to determine DNA methylation at one or more sites. Alternatively or additionally, DNA methylation may be detected using antibodies that distinguish between methylated and unmethylated sites, e.g., a methylation-specific anti CpG antibody.

Various methods may be used to identify polymorphic regions. In some embodiments, polymorphic regions may be identified by genotyping maternal nucleic acids. It is contemplated that genotype can be determined at any individual locus. Various genotyping assays or techniques are available in the art and can be adapted to practice the present invention.

Exemplary genotyping assays include, but are not limited to PCR, DNA fragment analysis, allele specific oligonucleotide (ASO) probes, DNA sequencing, and nucleic acid hybridization to DNA microarrays or beads. In some embodiments, suitable genotyping techniques include restriction fragment length polymorphism (RFLP), terminal restriction fragment length polymorphism (t-RFLP), amplified fragment length polymorphism (AFLP), and multiplex ligation-dependent probe amplification (MLPA).

Typically, genotyping assays suitable for the present invention are sufficiently sensitive to identify a substantial number of polymorphic regions between the mother and fetus. In some embodiments, more than 100, 500, 1,000, 2,000, 4,000, 6,000, 8,000, or 10,0000 polymorphic regions per chromosome are identified according to the present invention. In some embodiments, identified polymorphic regions are sequenced and the specific nature of the polymorphisms (e.g., SNPs) are determined.

Polymorphic regions are then characterized and/or quantified according to the present invention to identify differentially represented genomic regions in various maternal samples.

### Maternal Samples and Preparation Thereof

Any of a variety of maternal samples may be suitable for use with methods disclosed herein. Generally, any maternal samples containing both fetal and maternal nucleic acids may be used. Types of maternal samples include, but are not limited to, cells, tissue, whole blood, plasma, serum, urine, stool, saliva, cord blood, chorionic villus samples amniotic fluid, and transcervical lavage fluid. Cell cultures of any of the afore-mentioned maternal samples may also be used in accordance with inventive methods, for example, chorionic villus cultures, amniotic fluid and/or amniocyte cultures, blood cell cultures (e.g., lymphocyte cultures), etc.

In some embodiments, a suitable maternal sample is obtained from a pregnant woman by a non-invasive method. For example, a suitable maternal sample can be maternal blood, serum, plasma or amniotic fluid obtained from a pregnant woman. In particular embodiments, a suitable maternal sample is maternal blood (e.g., peripheral venous blood).

Suitable maternal samples may be obtained from individuals at various stages of pregnancy (e.g., during first, second or third trimester). In some embodiments, a suitable maternal sample is obtained during the first trimester, for example, between 4-13 weeks (e.g., between 6-13 weeks, between 8-13 weeks, between 9-13 weeks) of gestation. Typically, suitable maternal samples are obtained from individuals with normal pregnancy. In some embodiments, a suitable maternal sample is obtained from one individual. In some embodiments, a suitable maternal sample is a pooled sample from multiple individuals.

In some embodiments, total DNA is prepared from a maternal sample. In some embodiments, cell-free DNA is prepared from a maternal sample. Various methods and kits for preparing total DNA or cell-free DNA are available in the art and can be used to practice the present invention. For example, nucleic acid can be extracted from a maternal sample by a variety of techniques such as those described by Maniatis, et al., MOLECULAR CLONING: A LABORATORY MANUAL, Cold Spring Harbor, N.Y., pp. 280-281 (1982). Exemplary commercial kits that can be used to prepare cell-free DNA from maternal samples include, but are not limited to, QIAamp DNA Blood Midi Kit (Qiagen), High Pure PCR Template Preparation kit (Roche Diagnostics), and MagNA Pure LC (Roche Diagnostics).

Various amounts of maternal samples can be used. In some embodiments, a suitable maternal sample contains total or cell-free DNA with more than 1 (e.g., more than 2, 5, 10, 15, 20, 25, 50, 100, 200, 500, 1,000, 5,000, or 10,000) genomic equivalents. It is contemplated that 10-20 ml of maternal blood contains about 10,000 genome equivalents of total DNA during first trimester. Thus, in some embodiments, a suitable maternal sample may contain about 20 ml, 15 ml, 10 ml, 5 ml, 4 ml, 3 ml, 2 ml, 1 ml, 0.5 ml, 0.1 ml, 0.01 ml, or 0.001 ml of maternal blood.

In some embodiments, DNA preparations are randomly fragmented to produce fragments with suitable length for analysis. The nucleic acids to be characterized can be of variable lengths. For example, they can be at least 50 base pairs in length. In some embodiments, they can be 150 to 4000 base pairs in length. Various methods can be used to generate nucleic acid fragments such as sonication, restriction enzyme digestion, shot gun method, and others. Exemplary methods are described in U.S. patent application 2002/0190663 A1, published Oct. 9, 2003, the teachings of which are incorporated herein in their entirety.

In some embodiments, fragments may be further treated such that the ends of the different fragments all contain the same DNA sequence. Fragments with universal ends can then be amplified in a single reaction with a single pair of amplification primers. Fragments with universal ends may also be captured onto a solid support by universal capturing probes.

In some embodiments, to obtain unbiased quantification, no cloning or amplification is performed on nucleic acids in maternal samples before they are characterized by, e.g., sequencing, or hybridization.

It should be noted that, while the present description refers throughout to DNA, fetal RNA found in maternal blood may be analyzed as well. As described in Ng et al., “mRNA of placental origin is readily detectable in maternal plasma,” Proc. Nat. Acad. Sci., 100(8): 4748-4753, (2003), hPL (human placental lactogen) and hCG (human chorionic gonadotropin) mRNA transcripts were detectable in maternal plasma. For example, mRNA encoding genes expressed in the placenta and present on the chromosome of interest can be used. In this case, RNase H minus (RNase H—) reverse transcriptases (RTs) can be used to prepare cDNA for detection.

### Characterizing and Quantifying Genomic Regions

Various assays may be used to characterize and/or quantify fetal or maternal genomic regions of interest. For example, suitable methods may involve enumerating individual nucleic acid molecules/fragments containing a fetal or maternal genomic region of interest or measuring signal intensity changes for polymorphic probes (e.g., SNP specific probes) on a microarray (e.g., using array-based comparative genomic hybridization (aCGH) technology). Various methods may be used to enumerate individual nucleic acid molecules including, but not limited to, DNA sequencing (e.g., high throughput single molecule sequencing), digital PCR, bridge PCR, emulsion PCR, nanostring technology, among others. Exemplary methods are described in more detail below.

### Single Molecule Sequencing

In certain embodiments of the invention, methods comprise single molecule sequencing of nucleic acids in the maternal sample, for example, in order to characterize and/or quantify a fetal and/or maternal genomic region with certain sequence composition. In particular, single molecule sequencing techniques allow the evaluation of individual nucleic acid molecules with polymorphic nucleotides and obtaining sequence read counts attributable to distinct polymorphic regions.

Various single molecule sequencing methods have been described in the art and can be used to practice the present invention. See, e.g., Braslaysky et al., (2003), Proc. Natl. Acad. Sci., 100: 3960-64; Greenleaf et al., (2006), Science, 313: 801; Harris et al., (2008) Science, 320:106-109; Eid et al., (2009), Science, 323:133-138; Pushkarev et al., (2009), Nature Biotechnology, 27:847-850; Fan et al., (August 2008), Proc. Natl. Acad. Sci., Early Edition; the entire contents of each of which are incorporated by reference herein. Typically in single molecule sequencing techniques, nucleic acid fragments, which serve as templates during sequencing reactions, are immobilized to a solid support such that at least a portion of the nucleic acid fragment is individually optically-resolvable.

Solid supports suitable for the invention can be any solid surface to which nucleic acids can be covalently attached, such as, for example latex beads, dextran beads, polystyrene, polypropylene surface, polyacrylamide gel, gold surfaces, glass surfaces and silicon wafers. In some embodiments, solid support is a glass surface. In some embodiments, the solid support is a slide, e.g., a glass slide.

Means for attaching nucleic acids to a solid support as used herein refers to any chemical or non-chemical attachment method including chemically-modifiable functional groups. “Attachment” relates to immobilization of nucleic acid on solid supports by either a covalent attachment or via irreversible passive adsorption or via affinity between molecules (for example, immobilization on an avidin-coated surface by biotinylated molecules). Typically, the attachment is of sufficient strength that it cannot be removed by washing with water or aqueous buffer under DNA-denaturing conditions. “Chemically-modifiable functional group” as used herein refers to a group such as, for example, a phosphate group, a carboxylic or aldehyde moiety, a thiol, or an amino group.

In some embodiments, a solid support suitable for the invention has a derivatised surface. In some embodiments, the derivatised surface of the solid support is subsequently modified with bifunctional crosslinking groups to provide a functionalized surface, preferably with reactive crosslinking groups. “Derivatised surface” as used herein refers to a surface which has been modified with chemically reactive groups, for example amino, thiol or acrylate groups. “Functionalized surface” as used herein refers to a derivatised surface which has been modified with specific functional groups, for example the maleic or succinic functional moieties.

In some embodiments, each molecule of a nucleic acid fragment (which may comprise all or part of a fetal or maternal genomic region) is attached to the solid support at a distinct location. In some embodiments, nucleic acid fragments that are immobilized to a solid support are detectably labeled (e.g., labeled with a detectable moiety that can generate an optical signal). For example, the nucleic acid fragments may be annealed to an oligonucleotide primer that is detectably labeled. Locations of each single molecule on the solid support may be read by an instrument that detects the label (e.g., detectable moiety), and the locations of each molecule recorded. In some embodiments, the detectable label of the nucleic acid fragment is removed after locations are recorded. For example, in embodiments in which the detectable label comprises a fluorescent moiety, the detectable label may be removed by photobleaching the fluorescent moiety. Alternatively or additionally, the detectable label may be cleaved off of the nucleic acid fragment.

In some embodiments, capturing oligonucleotides are immobilized on the solid or semisolid support to facilitate capturing and immobilization of nucleic acid fragments (e.g., polynucleotides), as described further herein.

Sequencing reactions are performed using the immobilized nucleic acid fragments as templates. Primers are hybridized to the nucleic acid fragments to form a primer/template duplex. In some embodiments, nucleic acid fragments are modified to include adapters that are complementary to primers used. In some embodiments, primers are immobilized onto solid surfaces and nucleic acid fragments are attached to solid surfaces via their hybridization with primers.

In some embodiments, pyrosequencing (i.e., sequencing by synthesis) is performed. Specifically, template-dependent primer extension is performed in the presence of one or more nucleotides or nucleotide analogs (e.g., dNTPs) and one or more nucleic acid polymerases, under suitable conditions to allow extension of the primer by at least one base. Typically, nucleotides incorporated during sequencing reactions are detectably labeled (e.g., labeled with a detectable moiety that can generate an optical signal). Signal emanating from the label is detected and recorded; a particular signal may be associated with the identity of a particular nucleotide or nucleotide analog, thus revealing the identity of the corresponding complementary nucleotide on the template nucleic acid fragment. In some embodiments, detectable signals are removed and/or destroyed after a round of incorporation (e.g., as described herein), thus facilitating further extension and detection of labeled nucleotides or nucleotide analogs.

Sequencing can be optimized to achieve rapid and complete addition of the correct nucleotide to primers in primer/template complexes, while limiting the misincorporation of incorrect nucleotides. For example, dNTP concentrations may be lowered to reduce misincorporation of incorrect nucleotides into the primer. Kₘ values for incorrect dNTPs can be as much as 1000-fold higher than for correct nucleotides, indicating that a reduction in dNTP concentrations can reduce the rate of misincorporation of nucleotides. Thus, in some embodiments, the concentration of dNTPs in the sequencing reactions are approximately 5-20 μM.

In addition, relatively short reaction times can be used to reduce the probability of misincorporation. For example, for an incorporation rate approaching the maximum rate of about 400 nucleotides per second, a reaction time of approximately 25 milliseconds will be sufficient to ensure extension of 99.99% of primer strands.

Detectable moieties may be directly or indirectly incorporated into nucleotides, nucleotide analogs, polynucleotides, or other molecules as appropriate. Suitable detectable moieties include, among other things, fluorescent moieties and luminescent moieties. In some embodiments, a fluorescent moiety comprises a cyanine dye, e.g., cyanine-3 and/or cyanine 5. Examples of suitable detectable moieties are described further herein.

In some embodiments, single molecule sequencing is performed in a high-throughput fashion, e.g., with many sequencing reactions being performed in parallel. For example, a high throughput single molecule sequencing assay suitable for the invention may characterize up to thousands, millions, or billions of molecules simultaneously. Parallel sequencing reactions need not be performed synchronously; asynchronous reactions can be performed and are compatible with methods of the invention.

In accordance with methods of the invention, in some embodiments, individual sequence read counts are obtained that are attributable to a fetal or maternal genomic region. In some embodiments, attributing a sequence read count to a fetal or maternal genomic region is accomplished based on knowledge of polymorphisms between fetal and maternal nucleic acids and the detection of distinct label associated with polymorphic nucleotide.

In some embodiments, a large portion (e.g., more than 10%, 15%, 20%, 25%, 30%, 35%, 40%, 45%, 50%, 55%, 60%, 65%, 70%, 75%, 80%, 85%, 90%, 95%, 99%, or more than 99%) of the genome is sequenced. In some embodiments, at least one genomic region that is sequenced is covered on average at least 10 times (10× genome equivalents), that is, there are on average 10 reads or more of a given genomic region. In some embodiments, coverage is at least 20×, at least 30×, at least 40×, at least 50×, at least 60×, at least 70×, at least 80×, at least 90×, at least 100×, at least 110×, at least 120×, or more times. In some embodiments, coverage is 100 times (100× genome equivalents) or more.

In some embodiments, an unbiased nucleic acid sequencing method is employed. That is, the representation of a particular sequence among all the sequencing reads reflects the representation of the corresponding nucleic acid in the maternal sample. In some embodiments, unbiased nucleic acid sequencing is achieved at least in part by not amplifying the template nucleic acids before the sequencing reaction. In some embodiments, the template nucleic acid is also not amplified during the sequencing reaction. In some embodiments, unbiased DNA sequence uses bright fluorophores and laser excitation to detect pyrosequencing events from individual DNA molecules fixed to a surface, eliminating the need for amplification.

In some embodiments, unbiased nucleic acid sequencing is achieved at least in part by amplifying (during and/or before sequencing reactions) the template nucleic acids in a manner that ensures that all species in the population nucleic acids are amplified equally. For example, emulsion PCR may be used to amplify nucleic acids in an unbiased manner. See discussion the Emulsion PCR section.

Suitable reagents (e.g., nucleotides and/or nucleotide analogs, nucleic acid polymerases, etc.), solid supports, apparatuses, and methods of sequence analysis are known and have been described in the art. See, e.g., U.S. Pat. Nos. 7,169,560; 7,220,549; 7,276,720; 7,279,563; 7,282,337; 7,397,546; 7,424,371; 7,476,734; 7,482,120; 7,491,498; 7,501,245; 7,593,109; 7,635,562; 7,666,593; 7,678,894; and 7,753,095, the entire contents of each of which are herein incorporated by reference. Various commercially available kits such as True Single Molecule Sequencing (tSMS)^{TM} (Helicos) may be used to practice the present invention.

### Digital PCR

In some embodiments, digital PCR is used to characterize and quantify polymorphic fetal or maternal genomic regions. Typically, digital PCR involves amplifying a single DNA template from minimally diluted samples, therefore generating amplicons that are exclusively derived from one template and can be detected with different fluorophores to discriminate and count different polymorphic regions (e.g., fetal vs. maternal regions). Thus, digital PCR transforms the exponential, analog signals obtained from conventional PCR to linear, digital signals, allowing statistical analysis of the PCR product.

Digital PCR technology is well described in the art. See, Vogelstein B. and Kinzler K. W., (1999), Proc. Natl. Acad. Sci. USA, Vol. 96, pp 9236-9241; Pohl G. and Shih L. M., (2004), Expert. Rev. Mol. Diagn., 4(1), 41-47, the teachings of which are hereby incorporated by reference.

In some embodiments, DNA prepared from a maternal sample is first diluted onto multi-well (e.g., 96-well, 384-well) plates with one template per two wells on average (i.e., 0.5 template molecules (genomic equivalent) per well on average). To determine optimal dilution, DNA can be first quantified to determine the amount of genomic equivalents in the original maternal sample.

As the PCR products from the amplification of single template molecules are substantially homogeneous in sequence, a variety of techniques can be used to characterize the sequence content in each well. Typically, fluorescent probe-based detection methods are particularly useful. For example, to quantify fetal or maternal polymorphic regions, a pair of PCR primers and a pair of molecule beacons are designed for each SNP. Typically, molecule beacons are single-stranded oligonucleotides which contain a fluorescent dye and a quencher on their 5′ and 3′ ends, respectively. Both beacons are identical except for the nucleotide corresponding to the SNP and the fluorescent label (green or red). Typically, molecule beacons include a hairpin structure, which brings the fluorophore closer to the quencher, and do not emit fluorescence when not hybridized to a PCR product. Upon hybridization to their complimentary nucleotide sequences, the quencher is distanced from the fluorophore, resulting in increased fluorescence. Typically, the ratio of fluorescence intensity of two allele-specific beacons with either green or red fluorescence is calculated to determine the allele type in each individual well.

With hundreds or thousands of wells counted, the relative abundance of maternal and fetal (or paternal) alleles can be determined.

Various digital PCR methods, reagents, and apparatus are known in the art and can be adapted to practice the present invention. See, e.g., U.S. Pat. Nos. 6,143,496, 6,440,706, 6,753,147, and 7,704,687, the entire contents of each of which are herein incorporated by reference.

### Bridge PCR

In some embodiments, bridge PCR is used to characterize and/or quantify a fetal or maternal genomic region. Bridge PCR is also known as solid phase PCR or 2-dimensional PCR. In general, bridge PCR takes place on a solid surface or within a gel, thereby generating a large numbers of “polonies” (polymerase generated colonies) that can be simultaneously sequenced or hybridized with polymorphic probes.

In some embodiments, bridge PCR involves universal amplification reaction, whereby a DNA sample is randomly fragmented, then treated such that the ends of the different fragments all contain the same DNA sequence. For example, DNA fragments can be ligated to universal adapter sequences. Fragments with universal ends can then be amplified in a single reaction with a single pair of amplification primers. Typically, DNA fragments are first individually resolved on a surface, or within a gel, to the single molecule level at each reaction site prior to amplification, which ensures that the amplified molecules form discrete colonies that can then be further analyzed.

In some embodiments, these parallel amplification reactions occur on the surface of a “flow cell” (basically a water-tight microscope slide) which provides a large surface area for many thousands of parallel chemical reactions. The flow cell surface is coated with single stranded oligonucleotides that correspond to the sequences of the adapters ligated during the sample preparation stage. Single-stranded, adapter-ligated fragments are bound to the surface of the flow cell exposed to reagents for polymerase-based extension. Priming occurs as the free/distal end of a ligated fragment “bridges” to a complementary oligo on the surface. Various other solid surface may be used instead of the flow cell surface. For example, solid surface suitable for the invention may include, but are not limited to, latex beads, dextran beads, polystyrene, polypropylene surface, polyacrylamide gel, gold surfaces, glass surfaces and silicon wafers.

Various methods of bridge amplification are well known in the art. See, for example, U.S. Provisional Application Ser. No. 61/352,062, filed on Jun. 7, 2010, U.S. Pat. No. 7,115,400, U.S. Publication No. 20090226975, and Bing D. H. et al., “Bridge Amplification: A Solid Phase PCR System for the Amplification and Detection of Allelic Differences in Single Copy Genes,” Seventh International Symposium on Human Identification (available at the Promega website), all of which are hereby incorporated by reference.

Various methods can be used to characterize the sequence content of the amplified nucleic acids generated by bridge PCR. In some embodiments, millions polonies containing amplified nucleic acids may be sequenced by synthesis. For example, Illumina's Solexa Sequencing Technology may be adapted to characterize and quantify a fetal or maternal region accordingly to the present invention. For example, a solid surface containing millions of clusters may be subject to sequencing with automated cycles of extension and imaging. The first cycle of sequencing involves first of the incorporation of a single fluorescent nucleotide, followed by high resolution imaging of the entire surface. These images represent the data collected for the first base. Any signal above background identifies the physical location of a cluster (or polony), and the fluorescent emission identifies which of the four bases was incorporated at that position. This cycle is repeated, one base at a time, generating a series of images each representing a single base extension at a specific cluster. Base calls are derived with an algorithm that identifies the emission color over time. Thus, individual sequence read counts attributable to a specific fetal or maternal genomic region may be obtained.

In some embodiments, clusters containing amplified nucleic acids may be characterized by hybridization using fluorescent probe. For example, to distinguish and quantify fetal or maternal polymorphic regions, a pair of molecule beacons can be designed for each SNP. Typically, molecule beacons are single-stranded oligonucleotides which contain a fluorescent dye and a quencher on their 5′ and 3′ ends, respectively. Both beacons are identical except for the nucleotide corresponding to the SNP and the fluorescent label (green or red). Typically, molecule beacons include a hairpin structure, which brings the fluorophore closer to the quencher, and do not emit fluorescence when not hybridized to a PCR product. Upon hybridization to their complimentary nucleotide sequences, the quencher is distanced from the fluorophore, resulting in increased fluorescence. Typically, the ratio of fluorescence intensity of two allele-specific beacons with either green or red fluorescence is calculated to determine the allele type in each cluster. With hundreds or thousands of clusters counted, the relative abundance of maternal and fetal/paternal alleles can be determined.

### Emulsion PCR

In some embodiments, emulsion PCR is used to characterize and quantify a fetal or maternal genomic region. Typically, emulsion PCR can be used to generate small beads with clonally amplified DNA, i.e., each bead contains one type of amplicon generated from single molecule template by PCR. Exemplary emulsion PCR are described in Dressman et al, Proc. Natl. Acad. Sci. USA., 100, 8817 (Jul. 22, 2003) and Dressman et al. PCT publication W02005010145, “METHOD AND COMPOSITIONS FOR DETECTION AND ENUMERATION OF GENETIC VARIATIONS,” published 2005, Jan. 3, and hereby incorporated by reference for its description of a bead-based process.

For example, beads coated with capturing oligonucleotides (or colony primers) are mixed with nucleotides with complementary adaptor or tag sequences. An aqueous mix containing all the necessary components for PCR plus primer-bound beads and template DNA are stirred together with an oil/detergent mix to create microemulsions. The aqueous compartments (which may be illustrated as small droplets in an oil layer) contain an average of <1 template molecule and <1 bead. Different templates (maternal and fetal) may be pictured in one or less droplets to represent two template molecules whose sequences differ by one or many nucleotides. The microemulsions are temperature cycled as in a conventional PCR. If a DNA template and a bead are present together in a single aqueous compartment, the bead bound oligonucleotides act as primers for amplification.

Beads made of various materials and in various sizes can be used for the present invention. For example, suitable beads can be magnetic beads, plastic beads, gold particles, cellulose particles, polystyrene particles, to name but a few. Suitable beads can be microparticles in the size range of a few, e.g. 1-2, to several hundred, e.g. 200-1000 μm diameter. In some embodiments, commercially available controlled-pore glass (CPG) or polystyrene supports are employed as solid phase supports in the invention. Such supports come available with base-labile linkers and initial nucleosides attached, e.g. Applied Biosystems (Foster City, Calif.).

In some embodiments, beads containing clonally amplified nucleic acids may be characterized by pyrosequencing (i.e., sequencing by synthesis). For example, beads containing amplified DNA may be subject to a sequencing machine that contains a large number of picolitre-volume wells that are large enough for a single bead, together with enzymes needed for sequencing. In some embodiments, pyrosequencing uses luciferase to generate light as read-out, and the sequencing machine takes a picture of the wells for every added nucleotide and recorded. Sequence read counts attributable to fetal or maternal genomic regions may be obtained. Suitable sequencing machines are commercially available, including 454 Life Sciences's Genome Sequencer FLX.

### Single Molecule Hybridization With Barcoded Probes

In some embodiments, technology using single molecule hybridization with barcoded probes may be used to characterize and quantify a fetal or maternal genomic region. In general, such technology uses molecular “barcodes” and single molecule imaging to detect and count specific nucleic acid targets in a single reaction without amplification. Typically, each color-coded barcode is attached to a single target-specific probe corresponding to a genomic region of interest. Mixed together with controls, they form a multiplexed CodeSet. In some embodiments, two probes are used to hybridize each individual target nucleic acid. The Reporter Probe carries the signal; the Capture Probe allows the complex to be immobilized for data collection. After hybridization, the excess probes are removed and the immobilized probe/target complexes may be analyzed by a digital analyzer for data collection. Color codes are counted and tabulated for each target molecule (e.g., a fetal or maternal genomic region of interest). Suitable digital analyzers include nCounter® Analysis System provided by Nanostring Technologies.

Methods, reagents including molecular “barcodes” an apparatus suitable for nanostring technology are further described in U.S. App. Pub. Nos. 20100112710, 20100047924, 20100015607, the entire contents of each of which are herein incorporated by reference.

### Semiconductor Sequencing

In some embodiments, semiconductor sequencing methods are used to characterize and quantify a fetal or maternal genomic region. The term “semiconductor sequencing,” “semiconductor pH sensitive sequencing,” “replication detection sequencing,” “direct replication detection sequencing” and “semiconductor replication detection sequencing” as used herein are synonymous and refer generally to the methods of Pourmand and co-workers. See e.g., Pourmand et al., 2006, Proc. Natl. Acad. Sci. USA 103:6466-6470. Exemplary systems for semiconductor sequencing in this context include, e.g., Ion Torrent technology (Life Technologies, Guilford, Conn.). As with other methods of sequencing by synthesis known in the art and described herein, semiconductor sequencing methods are useful to sequence nucleic acid fragments immobilized on a solid support, i.e., a massively parallel array incorporating charge sensors to detect real-time release of proton during DNA replication. Typically, sample DNA is fragmented, e.g., 10-50, 50-150, 50-100, 100-200, 200-400, 400-4000 by sequences, preferably about 100 nucleotides. The sequences are prepared as a library with flanking adapters which are ligated or incorporated by designed PCR primers having the adapter sequences. The library fragments are then clonally amplified using emulsion PCR to form particles coated with template DNA. The particles are deposited on the massively parallel array, which is sequentially contacted with deoxynucleotide triphosphate (dNTP) in the presence of DNA polymerase under conditions suitable for DNA replication. Each incorporation of dNTP into the growing duplex DNA results in the release of a proton, resulting in a change in charge detectable by the charge sensors. Thus, a change in charge (i.e., change in pH) is a specific well of the massively parallel array indicates incorporation of a specific dNTP. No change in charge indicates that the specific dNTP was not incorporated. Multiple proton release (e.g., 2, 3, 4, or more) protons release indicates that a corresponding sequence of a specific dNTP was incorporated. Correlation of the change in charge of each well in the massively parallel array with the presence of a specific dNTP thus provides the sequence of the DNA sample.

Unidirectional sequencing requires only one fusion primer pair and will produce reads from only one end of the amplicon. Bidirectional sequencing can be conducted for optimal results, producing high quality reads from both ends and across the full length of the amplicons.

The length of the target regions can be optimized. For example, with a typical read length of 100 nucleotides, the first 20-25 nucleotides of sequence correspond to the target specific sequence of the PCR primers and will not produce informative data. Accordingly, in some cases, a target region of about 75 by is employed.

Depth of coverage requirements depend on the expected frequency of mutation with a sample and dictate the number of amplicons that are included given a fixed amount of sequence throughput per massively parallel array. For example, for germ-line mutations that follow standard Mendelian inheritance patterns, either 100% or 50% of the reads are expected to contain a given sequence variant. It is believed that in these cases an average depth of coverage of 100-200× provides a sufficient number of reads to detect variants with statistical confidence. For high confidence detection of somatic mutations present at variable and typically low frequencies in heterogeneous samples, e.g., heterogeneous cancer samples, deeper coverage of up to 1000-2000× is thought to be required.

Methods, reagents and apparatus are further described in the seminal work of Pourmand and co-workers, e.g., U.S. Pat. No. 7,785,785, incorporated herein by reference in its entirety and for all purposes.

### Detectable Entities

Any of a wide variety of detectable agents can be used in the practice of the present invention. Suitable detectable agents include, but are not limited to: various ligands, radionuclides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

In some embodiments, the detectable moiety is biotin. Biotin can be bound to avidins (such as streptavidin), which are typically conjugated (directly or indirectly) to other moieties (e.g., fluorescent moieties) that are detectable themselves.

In addition to exemplary detectable entities described in connection with various methods described herein, below are described some non-limiting examples of other detectable moieties.

### Fluorescent Dyes

In certain embodiments, a detectable moiety is a fluorescent dye. Numerous known fluorescent dyes of a wide variety of chemical structures and physical characteristics are suitable for use in the practice of the present invention. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′, 7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED™, SPECTRUM GREEN™, cyanine dyes (e.g., CY-3™, CY-5™, CY-3.5™, CY-5.5™, etc.), ALEXA FLUOR™ dyes (e.g., ALEXA FLUOR™ 350, ALEXA FLUOR™ 488, ALEXA FLUOR™ 532, ALEXA FLUOR™ 546, ALEXA FLUOR™ 568, ALEXA FLUOR™ 594, ALEXA FLUOR™ 633, ALEXA FLUOR™ 660, ALEXA FLUOR™ 680, etc.), BODIPY™ dyes (e.g., BODIPY™ FL, BODIPY™ R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™ 630/650, BODIPY™ 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al., (1995), Proc. Nat'l Acad. Sci. (USA), 92:4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described previously, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

### Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include betaglucuronidase, beta-D-glucosidase, urease, glucose oxidase, etc. An enzyme may be conjugated to a molecule using a linker group such as a carbodiimide, a diisocyanate, a glutaraldehyde, and the like.

### Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201T1, 212Bi, 213Bi, 211At, 153Sm, 177Lu).

In some embodiments, signal amplification is achieved using labeled dendrimers as the detectable moiety (see, e.g., Physiol Genomics, 3:93-99, 2000), the entire contents of which are herein incorporated by reference in their entirety. Fluorescently labeled dendrimers are available from Genisphere (Montvale, N.J.). These may be chemically conjugated to the oligonucleotide primers by methods known in the art.

### Determining Relative Abundance

Various method may be used to determine relative abundance of a fetal or maternal region. As used herein, the term “relative abundance” refers to an amount of a genomic region of interest as compared to a reference amount. Relative abundance can be determined as a ratio, a percentage, a change of fold, a normalized amount, among others.

Typically, to determine relative abundance, the amount of a fetal or maternal genomic region of interest is first measured or quantified by various methods including those described herein (e.g., single molecule sequencing, digital PCR, bridge PCR, emulsion PCR, nanostring technology or aCGH). This amount is then compared to a reference amount. A reference amount can be an amount indicative of the total amount of nucleic acid, the total amount of fetal or maternal nucleic acid in a relevant maternal sample (e.g., maternal blood). In this case, relative abundance of a fetal or maternal region is typically determined as a percentage of the relevant total amount of DNA.

In some embodiments, the amount of a fetal genomic region and the corresponding maternal genomic region are quantified. The relative abundance of a fetal genomic region may be determined by comparing the amount of the fetal genomic region to that of the corresponding maternal region. The relative abundance may be compared to a pre-determined threshold in order to determine if the fetal genomic region is differentially represented. Typically, in this case, a pre-determined threshold is indicative of an average ratio between fetal nucleic acid and maternal nucleic acid in a relevant maternal sample. A fetal genomic region is identified as overrepresented if the relative abundance is above a pre-determined threshold with statistical confidence.

In some embodiments, the relative abundance of a maternal genomic region may be determined by comparing the amount of the maternal genomic region to that of the corresponding fetal genomic region. The relative abundance may be compared to a predetermined threshold in order to determine if the maternal genomic region is differentially represented. Typically, in this case, a pre-determined threshold is indicative of an average ratio between maternal nucleic acid and fetal nucleic acid in a relevant maternal sample. A maternal genomic region is identified as underrepresented if the relative abundance is below a predetermined threshold with statistical confidence.

In some embodiments, relative abundance may be determined by comparing the quantified amount of a fetal or maternal genomic region to a reference amount indicative of average representation of fetal or maternal genomic region in a relevant maternal sample, respectively. Such average representation may be determined by quantifying the amount of a control region which is known not to be over or under represented in the maternal sample using the same assay performed simultaneously with the region of interest. In some embodiments, multiple control regions may be quantified and averaged to obtain a reference amount indicative of average representation. A suitable reference amount may also be a historical reference (i.e., an amount or result from an assay performed previously, or an amount or result that is previously known). In this case, if the quantified amount is statistically different (e.g., greater or less) than the reference amount, the fetal or maternal region of interest is identified as differentially represented (e.g., overrepresented or underrepresented).

In some embodiments, relative abundance may be determined by comparing the quantified amount of a fetal or maternal genomic region to a reference amount indicative of over representation of fetal or maternal genomic region in a relevant maternal sample. Such a reference may be determined by quantifying the amount of a control region which is known to be over represented in the maternal sample using the same assay performed simultaneously with the region of interest. In some embodiments, multiple overrepresented control regions may be quantified and averaged to obtain a reference amount indicative of over representation. A suitable reference amount may also be a historical reference (i.e., an amount or result from an assay performed previously, or an amount or result that is previously known). In this case, if the quantified amount is substantially the same or greater than the reference amount with statistical confidence, the fetal or maternal region of interest is identified as overrepresented.

In some embodiments, relative abundance may be determined by comparing the quantified amount of a fetal or maternal genomic region to a reference amount indicative of under representation of fetal or maternal genomic region in a relevant maternal sample. Such a reference may be determined by quantifying the amount of a control region which is known to be under represented in the maternal sample using the same assay performed simultaneously with the region of interest. In some embodiments, multiple underrepresented control regions may be quantified and averaged to obtain a reference amount indicative of under representation. A suitable reference amount may also be a historical reference (i.e., an amount or result from an assay performed previously, or an amount or result that is previously known). In this case, if the quantified amount is substantially the same or less than the reference amount with statistical confidence, the fetal or maternal region of interest is identified as underrepresented.

In some embodiments, relative abundance for every individual polymorphic genomic regions or loci are determined and a continuum model (e.g., a line or curve) may be denoted. The continuum may be compared to a baseline indicative of average representation of fetal or maternal nucleic acid, respectively, and any genomic regions or loci that deviate from the baseline with statistical confidence may be identified as differentially represented (e.g., over or under represented). In some embodiments, a reference amount indicative of the average representation of fetal nucleic acid in maternal circulation (e.g., maternal blood) may be about 3%, 5%, 10%, 15%, 20%, or 25%. In some embodiments, a reference amount indicative of the average representation of maternal nucleic acid in maternal circulation (e.g., maternal blood) may be about 97%, 95%, 90%, 85%, 80%, or 75%.

In some embodiments in which a genomic region is identified as overrepresented or underrepresented as compared to a reference amount, an “overrepresentation factor” or “underrepresentation factor” of the genomic region is determined. For example, if a fetal genomic region is determined to be overrepresented (e.g., 10%) as compared to a reference amount indicative of an average representation of fetal nucleic acids (e.g., 5%) in a maternal sample, the factor by which the observed amount of that fetal genomic region exceeds that of the reference amount is calculated as the “overrepresentation factor.” In this case, the overrepresentation factor is 2.

Typically, statistical tests are applied as described below or in accordance with other known methods in the art to determine where differences or similarity in amounts are statistically significant.

### Statistical Analyses

Typically, data are analyzed statistically to determine whether two values are the same or different (e.g., whether an amount of a genomic region is the same or different as a reference amount). A variety of statistical tests and measures of statistical significance are established in the art and may be used in accordance with the invention. Non-limiting examples of commonly used statistical tests for analyzing data that are evenly distributed and/or assumed to be evenly distributed (e.g., parametric tests) include the Student t-test (including one-sample t-tests, two-sample t-tests and matched pair t-tests) and analysis of variance (ANOVA; one-way and two-way or repeated-measures (e.g., N-way ANOVA)).

Non-limiting examples of commonly used statistical tests for analyzing data that are not evenly distributed include the Wilcoxon Rank-Sum test and the Mann Whitney U test.

Stringency (e.g., through cutoff values for p-values and/or q-values, as explained below) may be set according to a standard and/or may be set empirically for a given data set. The choice of a statistical test to use may depend on one or more factors including, but not limited to, distribution of the data, type of comparison being performed (e.g., experimental data to a reference value versus two sets of experimental data to each other) and relationship between samples (e.g., matched pairs (such as an experimental sample with a matched control) versus no relationship). In some embodiments, more than one statistical test is used, e.g., for confirmation purposes.

In some embodiments, a statistical test suitable for small sample sizes is used.

In some embodiments, analysis of relationships between multiple (e.g., more than two) groups is used. For example, an N-way ANOVA test (also known as repeated measures ANOVA test) generalizes a Student t-test to more than two groups. N-way ANOVA tests may be used in accordance with methods of the invention for more efficient comparison between multiple groups.

In some embodiments, multiple testing corrections are applied to adjust p-values derived from multiple statistical tests to correct errors that may arise from multiple testing (e.g., increased numbers of false positives or significant results). Multiple testing corrections typically involved recalculating probabilities from a statistical test that was repeated multiple times. In some embodiments, a Bonferroni correction is used. Multiple testing correction methods are known in the art. For a review of such methods, see, e.g., Noble, (2009), Nature Biotechnology, 27:1135-1137, the entire contents of which are incorporated herein by reference.

In some embodiments, a statistical test that involves analysis of the relationship(s) between two categorical variables is used.

For example, Fisher's exact test may be used to calculate exactly the significance of deviation from the null hypothesis; Fisher's exact test may be used in situations where the sample size is small. See, e.g., Weisstein, Eric W., “Fisher's Exact Test.” From Math World—A Wolfram Web Resource., available at the Wolfram.com website, the entire contents of which are herein incorporated by reference.

Two indicators of statistical significance are typically used to evaluate data. P-values indicate the probability of obtaining the values that were observed if the null hypothesis were not true. For example, the null hypothesis can be that a given fetal genomic region has an average representation. Lower p-values indicate statistical significance; i.e., increased likelihood that the null hypothesis is not true and should be rejected. Q-value indicates the false discovery rate, i.e. a measure of the proportion of false positives that occur when a particular test is considered significant. As with p-values, lower q-values indicate greater significance. In some embodiments, a p-value cutoff is used. In some embodiments, a q-value cutoff is used. In some embodiments, both a p-value and a q-value cutoff are used. In some embodiments, a p-value cutoff of p<0.05 is used. In some embodiments, a more stringent p-value cutoff, e.g., p<0.01, p<0.005, p<0.001, etc. is used. In some embodiments, a q-value of q<0.2 is used. In some embodiments, a more stringent q-value cutoff e.g., q<0.1, p<0.05, p<0.01, etc. is used. Any combination of p-value and q-value cutoff may be used in embodiments where both cutoffs are used, e.g., p<0.05 combined with q<0.2.

In some embodiments, quantified data are first normalized prior to statistical analysis. Typically, normalization is the process of isolating statistical error in repeated measured data. A normalization is sometimes based on a property. Quantile normalization, for instance, is normalization based on the magnitude (quantile) of the measures. In some embodiments, normalization refers to the division of multiple sets of data by a common variable in order to negate that variable's effect on the data, thus allowing underlying characteristics of the data sets to be compared: this allows data on different scales to be compared, by bringing them to a common scale. For example, an quantified amount of a fetal or maternal genomic region in a maternal sample may be normalized to the total amount of the genomic DNA in the sample to negate the effect of the amount variation in the starting material.

### Verification and Clinical Applications

In some embodiments, differentially represented fetal or maternal genomic regions may be compared across different biological individuals. Regions that are consistently over or under represented are identified and verified. Verification may be done by a repeat of the same techniques, and/or by additional techniques. For example, single molecule sequencing results may be validated, e.g., by digital PCR or by re-sequencing nucleic acids. Re-sequencing may be accomplished by the same methods and/or by other methods, e.g., Sanger sequencing. Over or under representation factors for each verified differentially represented region may be calculated.

Verified over or under represented fetal or maternal genomic regions may be identified for clinical applications based on their chromosomal locations, and associated genetic diseases, disorders or conditions. In some embodiments, over or under presentation factors and/or DNA sequence of the differentially represented regions are also provided. In some embodiments, the present invention provides a computer readable medium recorded with information relating to chromosomal locations, associated genetic diseases, disorders or conditions, over or under representation factors and/or DNA sequences of verified differentially represented fetal or maternal genomic regions.

Verified differentially represented fetal or maternal genomic regions may be used to develop or improve non-invasive pre-natal diagnosis of any genomic aberrations and associated genetic diseases, disorders and conditions associated with any of the differentially represented regions. As used herein, genetic aberrations may include, but are not limited to, nucleic acid base substitutions, amplifications, deletions, duplication, translocations, copy number variations, aneuploidy (e.g., polyploidy, trisomy, and the like) and mosaics. For example, characterization of relatively overrepresented fetal genomic regions in maternal circulation may provide more robust analysis of various genomic aberrations described herein, therefore, more accurate prenatal diagnosis of associated genetic diseases, disorders or conditions. In some embodiments, characterization of relatively overrepresented fetal genomic regions in maternal circulation may be used to develop non-invasive diagnostic assays with simplified, minimum or no enrichment or purification of fetal DNA. In some embodiments, characterization of relatively overrepresented fetal genomic regions in maternal circulation may be used to detect fetal abnormalities during early pregnancy (e.g., between 4-13 weeks, 4-9 weeks, or 4-6 weeks of gestation). In some embodiments, characterization of relatively overrepresented fetal genomic regions on chromosome 13, 14, 15, 16, 18, 21, 22, X, or any combination thereof, in maternal circulation may be used to detect chromosome abnormalities including, but not limited to, structural abnormalities, aneuploidy (e.g., polyploidy, trisomy, and the like), mosaics, mutations, and associated genetic diseases, disorders and conditions including, but not limited to, Turner's Syndrome, Down Syndrome (trisomy 21), Edward's Syndrome (trisomy 18), Patau Syndrome (trisomy 13), trisomy 14, trisomy 15, trisomy 16, trisomy 22, triploidy, tetraploidy, and sex chromosome abnormalities including but not limited to XO, XXY, XYY, and XXX.

### EXEMPLIFICATION

### Example 1

### Single Molecule Sequencing to Identify Overrepresented Regions of Fetal DNA in Maternal Blood

High-throughput single molecule sequencing is performed on cell-free DNA from maternal plasma from multiple individuals with an average of 100× or larger genome coverage.

Nucleic acids from maternal samples are fragmented and denatured into single strands. A polyA tail is added to each molecule. Single nucleic acid molecules are then captured on surfaces inside a flow cell, with each single molecule being captured at a distinct location.

A sequencing reaction is conducted using each molecule as a template without amplification. Fluorescently-labeled nucleotides (dCTP, dGTP, dATP, or dTTP) are added one at a time and incorporated into a growing complementary strand by a DNA polymerase. Unincorporated nucleotides are washed away. A laser is used to excited fluorophores on labeled nucleotides that were incorporated. The resulting emitted signals, and the positions of the signals, are detected and recorded in one or more images. Fluorescent labels of incorporated nucleotides are then removed by a highly efficient cleavage process that leaves behind the incorporated nucleotides, and then another nucleotide is added to continue the cycle. Nucleotide incorporation is thus tracked on each single molecule to determine the exact sequence of each individual DNA molecule.

With a fetal DNA fraction of 5%, on average, 95% of sequence reads are expected to be from maternal nucleic acids and 5% of sequence reads are expected to be from fetal nucleic acids. Statistical analysis of sequence read counts from fetal or maternal nucleic acids is performed to identify regions that are over-represented in cell-free fetal DNA.

For example, an over-represented locus may have 20 fetal sequence reads and 80 maternal sequence reads out of 100 total reads mapping to that locus's genomic location. Fisher's exact test is used to identify such regions based on the p-value of the observed counts as compared to the expected counts for a given average fetal fraction. Multiple testing correction is applied to increase the specificity of this approach. Regions of fetal DNA over-representation are then compared across different biological individuals and the most consistently overrepresented loci are selected for verification in a digital PCR assay or by other means.

### Example 2

Digital PCR to Characterize and/or Quantify Polymorphic Fetal or Maternal Genomic Regions

Digital PCR is employed to characterize and quantify polymorphic fetal or maternal genomic regions. Nucleic acids from minimally diluted maternal samples are fragmented and denatured into single strands, which are then amplified to generate amplicons that are exclusively derived from one template and can be detected with different fluorophores to discriminate and count different polymorphic regions (e.g., fetal vs. maternal regions). In this process, DNA prepared from a maternal sample is first diluted onto a 384-well multi-well plate with concentration adjusted to obtain about one template per two wells on average.

A pair of PCR primers and a pair of molecule beacons are designed for each SNP, the molecular beacons having a fluorescent dye and a quencher on their 5′ and 3′ ends, respectively. Both beacons are identical except for the nucleotide corresponding to an SNP and the fluorescent label (e.g., green or red). Upon hybridization to their complimentary nucleotide sequences, the quencher is distanced from the fluorophore, resulting in increased fluorescence. The ratio of fluorescence intensity of two allele-specific beacons with either green or red fluorescence is calculated to determine the allele type in each individual well. With hundreds or thousands of wells counted, the relative abundance of maternal and fetal (or paternal) alleles can be determined. Statistical analyses are conducted as described in Example 1.

### Example 3

Bridge PCR to Characterize and/or Quantify a Fetal or Maternal Genomic Region

Bridge PCR in a flow cell is conducted to characterize and/or quantify fetal or maternal genomic regions. A DNA sample is randomly fragmented then ligated to a universal adapter sequence. The flow cell surface is coated with single stranded oligonucleotides that correspond to the universal adapter sequence. Fragments with universal ends are then amplified in a single reaction with a single pair of amplification primers when single-stranded, adapter-ligated fragments bound to the surface of the flow cell are exposed to reagents for polymerase-based extension. Priming occurs as the free/distal end of a ligated fragment “bridges” to a complementary oligo on the surface, resulting in many copies of the DNA sample. Sequencing by synthesis is employed to sequence the DNA sample. Specifically, a surface of a flow cell containing millions of clusters is subject to sequencing with automated cycles of extension and imaging, using e.g., Illumina's Solexa Sequencing Technology. Each cycle of sequencing involves the steps of incorporating a single fluorescent nucleotide followed by high resolution imaging of the entire surface. Any signal above background identifies the physical location of a cluster (or polony), and the fluorescent emission identifies which of the four bases was incorporated at that position. This cycle is repeated, one base at a time, generating a series of images each representing a single base extension at a specific cluster. Base calls are derived with an algorithm that identifies the emission color over time. Thus, individual sequence read counts attributable to a specific fetal or maternal genomic region are obtained. Statistical analyses are conducted as described in Example 1.

### Example 4

Emulsion PCR to Characterize and/or Quantify a Fetal or Maternal Genomic Region

Emulsion PCR is used to characterize and quantify a fetal or maternal genomic region. Small beads are generated with clonally amplified DNA, wherein each bead contains one type of amplicon generated from single molecule template by PCR. Beads coated with capturing oligonucleotides are mixed with nucleotides with complementary adaptor or tag sequences. An aqueous mix containing all the necessary components for PCR plus primer-bound beads and template DNA are stirred together with an oil/detergent mix to create microemulsions. The aqueous compartments contain an average of <1 template molecule and <1 bead. The microemulsions are temperature cycled as in a conventional PCR. If a DNA template and a bead are present together in a single aqueous compartment, the bead bound oligonucleotides act as primers for amplification.

Beads made of various materials, e.g., magnetic beads, plastic beads, gold particles, cellulose particles, polystyrene particles and the like, and in various sizes are used for emulsion PCR. Suitable beads can be microparticles in the size range of a few, e.g. 1-2, to several hundred, e.g. 200-1000 μm diameter. In some embodiments, commercially available controlled-pore glass (CPG) or polystyrene supports are employed as solid phase supports in the invention. Such supports come available with base-labile linkers and initial nucleosides attached, e.g. Applied Biosystems (Foster City, Calif.).

Beads containing clonally amplified nucleic acids are characterized by pyrosequencing as known in the art. Sequence read counts attributable to fetal or maternal genomic regions are thus obtained. Suitable sequencing machines include the 454 Life Sciences's Genome Sequencer FLX.

Statistical analyses are conducted as described in Example 1.

### Example 5

Single Molecule Hybridization with Barcoded Probes to Characterize and/or Quantify a Fetal or Maternal Genomic Region

Single molecule hybridization with barcoded probes, as known in the art, is used to characterize and quantify fetal or maternal genomic regions. Accordingly, molecular barcodes and single molecule imaging are useful to detect and count specific nucleic acid targets in a single reaction without amplification. Each color-coded barcode is attached to a single target-specific probe corresponding to a genomic region of interest. Two probes (i.e., the so-called “Reporter” and “Capture” probes) are used to hybridize each individual target nucleic acid. The Reporter Probe carries the signal, and the Capture Probe allows the complex to be immobilized for data collection. After hybridization, excess probes are removed and the immobilized probe/target complexes are analyzed by a nCounter® Analysis System digital analyzer (Nanostring Technologies, Seattle Wash.) for data collection. Color codes are counted and tabulated for each target molecule (e.g., a fetal or maternal genomic region of interest).

Statistical analyses are conducted as described in Example 1.

### Example 6

Semiconductor Sequencing to Characterize and/or Quantify a Fetal or Maternal Genomic Region

Semiconductor sequencing is used to characterize and quantify a fetal or maternal genomic region. Sample DNA from maternal samples is fragmented and denatured into single strands having about 100 bp. A library is constructed incorporating bidirectional flanking adapters which are incorporated by designed PCR primers having the adapter sequences. The library fragments are clonally amplified using emulsion PCR to form particles coated with template DNA. The particles are deposited on a massively parallel array incorporating charge sensors to detect real-time release of proton during DNA replication. The massively parallel array is sequentially contacted with each of the deoxynucleotide triphosphates (dNTPs) in turn in the presence of DNA polymerase under conditions suitable for DNA replication. Each incorporation of dNTP into the growing duplex DNA results in the release of a proton, resulting in a change in charge detectable by the charge sensors. Correlation of the change in charge of each well in the massively parallel array with the presence of a specific dNTP provides the sequence of the DNA sample.

Statistical analyses are conducted as described in Example 1.

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

### INCORPORATION OF REFERENCES

All publications and patent documents cited in this application are incorporated by reference in their entirety to the same extent as if the contents of each individual publication or patent document were incorporated herein.

What is claimed is:

1. A method of identifying differentially represented fetal or maternal genomic regions in a maternal sample, comprising steps of
quantifying a fetal or maternal genomic region present in a maternal sample;
determining relative abundance of the fetal or maternal genomic region as compared to a reference amount, thereby determining if the fetal or maternal genomic region is differentially represented in the maternal sample;
wherein the fetal or maternal genomic region does not correspond to an aneuploidic region.

2. The method of claim 1, wherein the reference amount is indicative of an average representation of fetal or maternal nucleic acid in a maternal sample.

3. The method of claim 2, wherein the step of determining relative abundance comprises comparing the quantified amount to the reference amount and further wherein the fetal or maternal genomic region is identified as differentially represented in the maternal sample if the quantified amount is different than the reference amount with statistical confidence.

4. The method of claim 1, wherein the reference amount is indicative of an overrepresentation of fetal or maternal nucleic acid in a maternal sample.

5. The method of claim 4, wherein the step of determining relative abundance comprises comparing the quantified amount to the reference amount and further wherein the fetal or maternal genomic region is identified as overrepresented in the maternal sample if the quantified amount is substantially the same as or greater than the reference amount with statistical confidence.

6. The method of claim 1, wherein the reference amount is indicative of an underrepresentation of fetal or maternal nucleic acid in a maternal sample.

7. The method of claim 6, wherein the step of determining relative abundance comprises comparing the quantified amount to the reference amount and further wherein the fetal or
maternal genomic region is identified as underrepresented in the maternal sample if the quantified amount is substantially the same as or less than the reference amount with statistical confidence.

8. The method of claim 1, wherein the method quantifies a fetal genomic region.

9. The method of claim 8, wherein the reference amount is indicative of an average representation of fetal nucleic acid in the maternal sample.

10. The method of claim 9, wherein the average representation of fetal nucleic acid is about 5%.

11. The method of claim 8, wherein the fetal genomic region is identified as overrepresented in the maternal sample if the amount quantified is above the reference amount.

12. The method of claim 1, wherein the method quantifies a maternal genomic region.

13. The method of claim 12, wherein the reference amount is indicative of an average representation of maternal nucleic acid in the maternal sample.

14. The method of claim 13, wherein the average representation of maternal nucleic acid is about 95%.

15. The method of claim 12, wherein the maternal genomic region is identified as underrepresented in the maternal sample if the amount quantified is below the reference amount.

16. The method of claim 1, wherein the quantifying step comprises quantifying a fetal genomic region and the corresponding maternal genomic region.

17. The method of claim 16, wherein the determining step comprises determining the relative abundance of the fetal genomic region by comparing the quantified amount of the fetal genomic region to the quantified amount of the corresponding maternal genomic region.

18. The method of claim 1, wherein the fetal genomic region is distinctively detectable from the corresponding maternal genomic region.

19. The method of claim 1, wherein the fetal genomic region comprises a paternally contributed sequence.

20. The method of claim 18, wherein the fetal genomic region comprises a sequence distinct from the corresponding maternal genomic region.

21. The method of claim 20, wherein the fetal genomic region comprises at least one polymorphic nucleotide distinct from the corresponding maternal genomic region.

22. The method of claim 18, wherein the fetal genomic region comprises a methylation pattern that is distinct from the corresponding maternal genomic region.

23. The method of claim 18, wherein the fetal genomic region comprises copy number variation (CNV) as compared to the corresponding maternal genomic region.

24. The method of claim 1, wherein the method quantifies multiple fetal or maternal genomic regions simultaneously.

25. The method of claim 1, wherein the method further comprises a step of first preparing total DNA from the maternal sample.

26. The method of claim 1, wherein the method further comprises a step of first preparing cell free DNA from the maternal sample.

27. The method of claim 1, wherein the method further comprises a step of first generating nucleic acid fragments comprising the fetal or maternal genomic region to be quantified.

28. The method of claim 1, wherein the maternal sample is selected from the group consisting of cells, tissue, whole blood, plasma, serum, urine, stool, saliva, cord blood, chorionic villus sample, chorionic villus sample culture, amniotic fluid, amniotic fluid culture, transcervical lavage fluid, and combinations thereof

29. The method of claim 28, wherein the maternal sample is maternal blood.

30. The method of claim 1, wherein the maternal sample is obtained from one individual.

31. The method of claim 1, wherein the maternal sample is obtained from multiple individuals.

32. The method of claim 1, wherein the quantifying step comprises a DNA sequencing step.

33. The method of claim 32, wherein the DNA sequencing step comprises a high-throughput single molecule sequencing step.

34. The method of claim 32, wherein the DNA sequencing step comprises an unbiased DNA sequencing step.

35. The method claim 32, wherein the DNA sequencing step covers greater than 100 genomic equivalence.

36. The method of claim 32, wherein the DNA sequencing step comprises a step of labeling the fetal or maternal genomic region with an optical signal.

37. The method of claim 36, wherein the optical signal is selected from fluorescent and/or luminescent signal.

38. The method of claim 37, wherein the fluorescent signal is generated by Cyanine-3 and/or Cyanine-5.

39. The method of claim 32, wherein the method further comprises a step of capturing nucleic acid molecules comprising the fetal or maternal genomic region onto a solid surface prior to the sequencing step.

40. The method of claim 32, wherein the quantifying step comprises obtaining individual sequence read counts attributable to the fetal or maternal genomic region.

41. The method of claim 40, wherein the quantifying step further comprises comparing the individual sequence read counts attributable to the fetal genomic region to the individual sequence read counts attributable to the corresponding maternal genomic region.

42. The method of claim 1, wherein the quantifying step comprises a step of performing digital PCR.

43. The method of claim 1, wherein the quantifying step comprises a step of performing bridge PCR.

44. The method of claim 1, wherein the quantifying step comprises a step of hybridizing individual nucleic acid molecules using probes labeled with nanoreporters that specifically bind to the fetal or maternal genomic region.

45. The method of claim 1, wherein the quantifying step comprises a step of performing array-based comparative genomic hybridization (aCGH).

46. The method of claim 45, wherein the aCGH step uses probes that specifically bind to the fetal or maternal genomic region.

47. The method of claim 46, wherein the probes are labeled with optical signal.

48. The method of claim 47, wherein the optical signal is selected from fluorescent and/or luminescent signal.

49. The method of claim 47, wherein the aCGH step comprises determining the level of signal attributable to the fetal or maternal genomic region.

50. The method of claim 1, wherein the statistical confidence is determined by N-way ANOVA, Student t-test, or Fisher's exact test.

51. The method of claim 1, wherein multiple testing corrections are performed on the statistical confidence.

52. The method of claim 1, wherein the method further comprises determining an overrepresentation factor of the fetal genomic region.

53. The method of claim 1, wherein the method further comprises comparing the identified differentially represented fetal or maternal genomic region across different individuals.

54. The method of claim 1, wherein the method further comprises validating the identified differentially represented fetal or maternal genomic region by digital PCR or re-sequencing.

55. A method of non-invasive diagnosis comprising a step of characterizing an overrepresented fetal genomic region identified using the method claim 1.

56. A method of identifying fetal genomic regions normally overrepresented in a maternal sample, comprising steps of
characterizing a fetal genomic region and corresponding maternal genomic region in a maternal sample;
determining relative abundance of the fetal genomic region as compared to the corresponding maternal genomic region; and
identifying the fetal genomic region as overrepresented in the maternal sample if the relative abundance determined is above a pre-determined threshold with statistical confidence, wherein the fetal genomic region is not an aneuploidic region.

57. A method of identifying maternal genomic regions normally underrepresented in a maternal sample, comprising steps of
characterizing a maternal genomic region and corresponding fetal genomic region in a maternal sample;
determining relative abundance of the maternal genomic region as compared to the corresponding fetal genomic region; and
identifying the maternal genomic region as underrepresented in the maternal sample if the relative abundance determined is below a pre-determined threshold with statistical confidence, wherein the corresponding fetal genomic region is not an aneuploidic region.

58. A method of identifying fetal genomic regions normally overrepresented in a maternal sample, comprising steps of
characterizing a fetal genomic region in a maternal sample;
determining relative abundance of the fetal genomic region as compared to a reference; and
identifying the fetal genomic region as overrepresented in the maternal sample if the relative abundance determined is above a pre-determined threshold with statistical confidence, wherein the fetal genomic region is not an aneuploidic region.

59. The method of claim 58, wherein the reference is indicative of an average representation of fetal nucleic acid in a maternal sample.

60. A method of identifying maternal genomic regions normally underrepresented in a maternal sample, comprising steps of
characterizing a maternal genomic region in a maternal sample;
determining relative abundance of the maternal genomic region as compared to a reference; and
identifying the maternal genomic region as underrepresented in the maternal sample if the relative abundance determined is below a pre-determined threshold with statistical confidence,
wherein the maternal genomic region does not correspond to an aneuploidic region.

61. The method of claim 60, wherein the reference is indicative of an average representation of maternal nucleic acid in a maternal sample.
